(19) United States
(12) Patent Application Publication
(10) Pub. No.: US 2004/0030270 A1
(43) Pub. Date: Feb. 12, 2004

(54) Compression garment with electro-stimulation

(76) Inventor: James P. Johnson, Gainesville, GA (US)

Correspondence Address: GARDNER GROFF, P.C., PAPER MILL VILLAGE, BUILDING 23, 600 VILLAGE TRACE, SUITE 300, MARIETTA, GA, 30067, US

(21) Appl. No.: 10/460,084

(22) Filed: Jun. 12, 2003

Related U.S. Application Data
(60) Provisional application No. 60/387,963, filed on Jun. 12, 2002.
(60) Provisional application No. 60/476,351, filed on Jun. 6, 2003.

(51) Int. Cl. A61N 1/32

(52) U.S. Cl. 601/15; 601/21; 601/22; 601/151; 607/3; 607/144; 607/149

(57) ABSTRACT

A therapeutic garment combining compression therapy such as gradient compression of a body part or tissue of a human or animal subject with electrical or other form of delivered-energy stimulation. Garment electrodes have conductive portions contiguous or integral with elastic portions for delivering electrical stimulation to a treated area that is under compression. The combination of electrotherapy in conjunction with compression can help reduce edema and promote optimum blood circulation and improved healing.

### CROSS-REFERENCE TO RELATED APPLICATIONS

This application claims the benefit of U.S. Provisional Patent Application Serial No. 60/387,963, filed Jun. 12, 2002, and of U.S. Provisional Patent Application Serial No. [not yet available—attorney docket no. 2P04.1-020], filed Jun. 6, 2003, which are hereby incorporated herein by reference in their entireties for all purposes. U.S. Pat. Nos. 4,554,923, 4,664,118 and 5,374,283 are also hereby incorporated herein by reference.

### FIELD OF THE INVENTION

The present invention relates generally to therapeutic garments, and more particularly to a garment for providing compression therapy in combination with electrical stimulation to nerve,- muscle, skin, circulatory, and other tissues in a human or animal subject.

### BACKGROUND OF THE INVENTION

Compression therapy provides many benefits for human and/or animal patients with venous disease or related and similar conditions. Compression reduces the effective diameter of the veins, thereby increasing flow velocity and decreasing the chance of thrombosis. It also activates the fibrinolytic activity in the blood, with the same results. It reduces filtration of fluid out of the intravascular space and improves lymphatic flow, thereby reducing edema. Compression is also anti-inflammatory, yielding improvement in pain and swelling. Some of the effects of compression have been found to last for a period of time, even after the compression is removed.

Compression garments are particularly useful in promoting stability, increasing blood flow and reducing edema in the treatment of various injuries or diseases to the lower extremities, as well as to the ankle, knee, hand/wrist, elbow, etc. Graduated compression reduces reflux and improves venous outflow, thus decreasing venous pressure at rest and with ambulation. For example, gradient compression socks and stockings are useful for treating poor circulation in the leg. The function of gradient compression hosiery is to deliver pressure at the ankle that gradually decreases as it extends up the leg, helping to move blood back to the heart. This activity reduces blood pooling in the legs.

Elastic compression is generally easy to apply, and can be provided using aesthetically acceptable devices. For patients with symptoms such as aching or pain due to telangiectatic veins, varicose veins, or chronic venous insufficiency, elastic compression stockings may reduce the severity of their symptoms and retard the progression of their disease. However, it must be recognized that some patients may actually be harmed by compression, particularly if elastic stockings are not fit properly. Fitting must include measurements of the ankle, calf, thigh, and hop as appropriate to the length of the stocking. Stockings that are sized simply by the height and weight of the patient may result in the production of a harmful pressure gradient and should not be used for patients with venous disease.

There is one other potential disadvantage of elastic stockings. Venous emptying has two phases: the first is the working phase, during which the muscles contract and the blood is forced through the venous valves, up through the deep veins, toward the heart. During the next phase, the resting phase, the muscles relax and the pressure in the deep veins decreases, allowing blood to move from the superficial veins and through the perforating veins in the deep veins. However, as the muscles swell during their contraction, they expand the stocking. During muscle relaxation, the elastic in the stocking recoils and creates inward pressure on the leg. Since it is important for the pressure within the deep venous system to be low in order to facilitate the movement of the blood from the superficial venous system, the elastic recoil may reduce the filling of the deep veins. In this way, the efficiency of the musculovenous pump is compromised in some patients by the use of elastic compression stockings.

Electrical stimulation has separately been found to provide therapeutic benefit to various biological tissues. For example, transcutaneous electrical nerve stimulation (TENS) has been used to treat neuromuscular injuries and other conditions by stimulation of muscle and nerve tissue. Electrical stimulation has also been found to increase circulation, promoting faster healing of injuries and preventing adverse conditions resulting from poor circulation. For example, many diabetics suffer from poor circulation in their extremities, which can be improved by electrical stimulation. Foot ulceration caused by poor circulation often progresses to more severe tissue damage and even loss of limb in many subjects. These conditions are commonly aggravated as a result of venous stasis or impaired function of venous valves in the extremities.

Thus it can be seen that compression therapy, and more particularly compression therapy using gradient compression, can be beneficial to human and animal subjects with various conditions. Needs exist, however, for improvements to known compression garments. In addition, the applicant has now discovered that electrostimulation provided in combination with compression therapy, and more particularly compression therapy using gradient compression, can increase the therapeutic benefit to human and animal subjects, as compared to compression therapy or electrical stimulation alone, by stimulating enhanced circulation in the region under compression. However, known devices for compression therapy typically do not enable electrostimulation, and known devices for electrostimulation typically do not enable compression therapy. Thus it can be seen that further need exists for devices and methods for delivering compression therapy, and more particularly compression therapy using gradient compression, in combination with electrostimulation of the region under compression. It is to the provision of devices and methods meeting these and other needs that the present invention is primarily directed.

### SUMMARY OF THE INVENTION

The present invention provides an improved compression garment, such as a stocking, sleeve, wrap, glove, or other type of garment to be worn over one or more body parts of a human or animal subject. At least a portion of the garment preferably comprises a conductive material for providing electrical stimulation. In further preferred aspects, the garment optionally provides gradient compression.

In one aspect, the present invention is a therapeutic garment comprising at least one compressive portion, and at least one electrically conductive portion. In another aspect, the invention is a therapeutic garment comprising at least one gradiently compressive portion, and at least one electrically conductive portion.

In another aspect, the invention is a therapeutic garment, at least a portion of which comprises an elastic knit of electrically-conductive fibers, the elastic knit being configured to apply gradient compression when applied to a treated area.

In yet another aspect, the invention is a therapeutic garment including a first conductive portion comprising an electrically-conductive elastic knit material, a second conductive portion comprising an electrically-conductive elastic knit material, and a non-conductive portion between the first and second conductive portions. The elastic knit of the first conductive portion is preferably configured to apply a greater degree of compression to a treated area than the elastic knit of the second conductive portion.

In still another aspect, the invention is a method of improving circulation in a human or animal subject, said method comprising wearing a compressive garment on a body portion of the subject, and applying electrical stimulation to at least a portion of the compressive garment. In another aspect, the invention is a method of improving circulation in a human or animal subject, said method comprising wearing a gradiently compressive garment on a body portion of the subject, and applying electrical stimulation to at least a portion of the compressive garment.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWING FIGURES

FIG. 1 shows a therapeutic garment for compression and electrostimulation according to an embodiment of the present invention.

FIG. 2 shows an example electrostimulation pulse wave pattern for use with an embodiment of the method and garment of the present invention.

### DETAILED DESCRIPTION OF PREFERRED EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

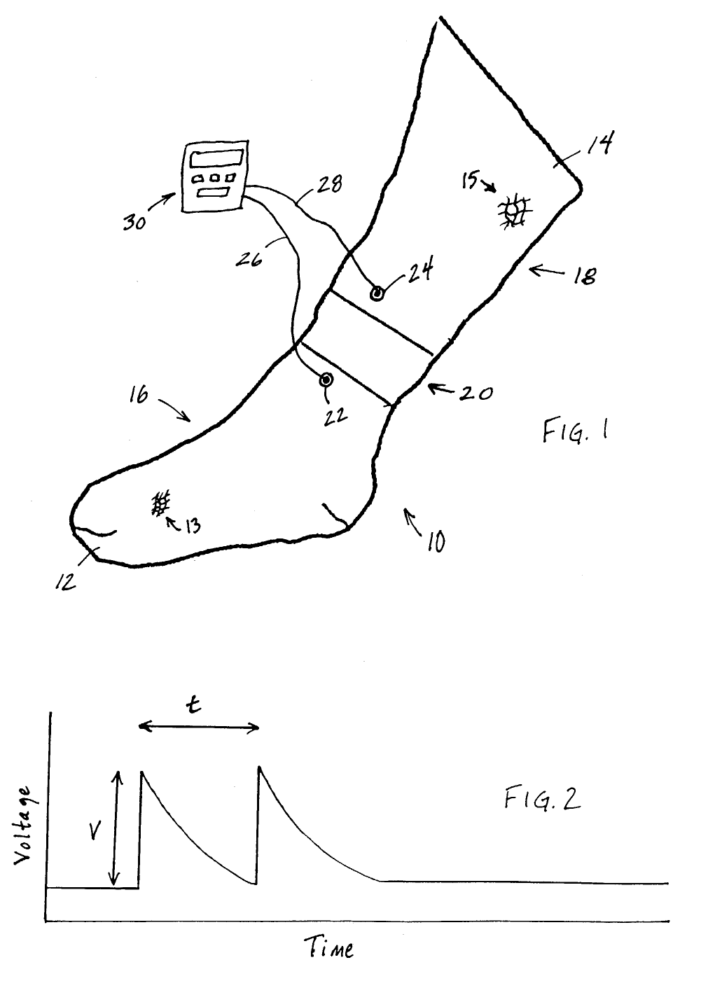

FIG. 1 shows a garment 10 according to one form of the present invention. The garment is depicted as a stocking, but other embodiments of the invention include a sleeve, wrap, glove, or other type of garment to be worn over one or more body parts of a human or animal subject. The garment 10 is preferably at least partially elastic, and is sized and shaped to provide compression when worn on the intended body part(s) of the subject. For example, garments according to the present invention can be provided in a range of sizes and garment configurations for subjects of virtually any anticipated size and shape, or can be custom fabricated to the specifications of an individual subject.

In further preferred and optional embodiments, at least a portion of the garment 10 is sized, shaped and configured to provide gradient compression when worn by the subject. For example, a stocking can be knit to provide a greater degree of compression toward the subject's toes, and progressively lesser degrees of compression toward the upper open calf end of the stocking, when worn. Gradient compression can be achieved, for example, by varying the fiber content and/or knit characteristics of the garment 10, by variation of the fiber tension within the knit, and/or by tapering the shape of the garment to provide a tighter fit (i.e., greater compression) in one portion and a looser fit (i.e., less compression) in another portion. For example, the toe end 12 of the stocking 10 can be fabricated to produce a greater degree of compression than the calf or knee end 14. In the depicted embodiment, the garment comprises a first electrically-conductive knit of fibers 13, which is relatively tightly knit to provide a greater degree of compression toward the wearer's toes, and a second electrically-conductive knit of fibers 15, which is relatively loosely knit to provide a lesser degree of compression toward the wearer's calf.

The garment 10 of the present invention preferably comprises one or more electrically conductive portions. For example, fibers, loops, rings, strips, panels, links or other elements of silver, copper, gold, other metals, and/or other conductive materials can be incorporated into the garment. Alternatively, non-conductive fibers or other elements of the garment 10 can be impregnated with a conductive material, coated with a conductive material, or otherwise rendered conductive. In other embodiments of the invention, a garment 10 or a portion thereof is formed of non-conductive knitted fabric or other non-conductive material and is plated on one or more faces with a conductive material, or otherwise rendered electrically conductive. For example, in the depicted embodiment, the garment 10 comprises a first electrically conductive portion 16 proximal the toe end, and a second electrically conductive portion 18 proximal the calf end, with the first and second electrically conductive portions separated by a non-conductive portion 20 proximal an ankle region of the stocking.

In an alternate embodiment, the garment is a glove, and the first conductive portion is proximal a finger portion of the glove and the second conductive portion is proximal a wrist or forearm portion of the glove. The glove provides compression to an underlying treated area to which it is applied, and more preferably provides gradient compression with a greater degree of compression at the fingers than at the wrist or forearm. In another alternate embodiment, therapeutic garment is a sleeve, the first conductive portion being proximal a first open end of the sleeve and the second conductive portion being proximal a second open end of the sleeve, wherein the first open end of the sleeve is smaller than the second open end of the sleeve. The sleeve provides compression to an underlying treated area to which it is applied, and more preferably provides gradient compression with a greater degree of compression toward the first open end than toward the second open end.

Each electrically conductive portion of the garment 10 preferably comprises an electrical connector coupling. For example, a first metallic snap connector 22 is provided on the first electrically conductive portion 16, and a second metallic snap connector 24 is provided on the second electrically conductive portion 18. Conductive leads 26, 28 are preferably provided for connection of the garment 10 to output terminals of an electrical stimulator 30, which delivers stimulation energy to the garment. Alternatively, the output terminals of the stimulator 30 are configured for direct connection with the garment 10.

An example embodiment of the stocking electrode 10 with gradient compression comprises an over-the-calf stocking with conductive silver nylon fibers plated in the upper and lower segments 16, 18, inside the stocking. The conductive ends are separated by a non-conductive band 20 approximately 1″-3″ (approximately 2.5 cm-7.5 cm) wide. Fiber contents of an example embodiment of a garment 10 according to the present invention are approximately: 56% Thermostat polyester, 38% silver, 4% spandex, and 2% nylon. The conductive portions of the garment are contiguous or integral with elastically compressive portions thereof, so that electrical stimulation is delivered to the treated area that is under compression.

In other embodiments of the invention, a conductive garment without compression features may be applied to a body part in combination with a standard non-conductive compression or gradient compression garment, for example by layering the compression garment over the conductive garment. For ease of use, the compression garment is optionally permanently or releasably attached to the conductive garment in their layered configuration, as by stitching, adhesive, snaps, buttons, hook-and-loop materials, and/or other attachment means.

In contrast to previously known therapeutic garments and methods, the application of electrical stimulation or electrotherapy in combination with compression, and more preferably with gradient compression, has been found to provide greatly improved therapeutic results by enhancing blood flow in the region(s) under compression, enhancing the reduction of edema, inflammation, and pain. During the resting phase of circulation, electrical stimulation further assists in the necessary inflow of blood into the deep venous system. In addition, compression, and more preferably gradient compression, can be used in combination with electrotherapy in treating other injured body parts such as the knee, ankle, hand/wrist and elbow where there is a need to reduce edema and improve circulation. Incorporating compression with other garments, including without limitation the Silver-Thera™ Knee Sleeve, Ankle Sleeve, Hand/Wrist Glove and Elbow Sleeve garment electrodes of Prizm Medical Inc. of Duluth, Ga., promotes a unique and more effective therapy.

The Silver-Thera™ garment electrodes incorporate a patented technology (see U.S. Pat. Nos. 4,554,923; 4,664,118 and 5,374,283 which are incorporated herein by reference), constructed so that there are two conductive regions or electrodes 16, 18 incorporated in the medical garment device, separated by a neutral zone 20 of nonconductive fibers. Powered by an electrical stimulator such as the Micro-Z™ neuromuscular stimulator of Prizm Medical Inc. of Duluth, Ga., or other energy source, the garment electrodes provide electrical energy accelerating and enhancing microcirculation. Embodiments of the present invention combine this Silver-Thera™ garment electrode technology with compression, and optionally with gradient compression, according to the disclosure herein.

The stimulator 30 preferably delivers electrical stimulation from an energy source such as a battery to the garment 10 or other type of electrode according to a prescribed stimulation sequence or regimen (e.g., controlling parameters of the delivered energy including: current, voltage, waveform, frequency, duration, cycle parameters, etc.). For example, delivery of microamperage stimulation in a high-volt pulse galvanic waveform or twin-peak monophasic waveform, as shown in FIG. 2, with for example, a 100V peak voltage V and 100 microsecond peak-to-peak pulse times t, has been found to provide suitable therapeutic results. Further embodiments of the invention comprise the delivery of noise (e.g., sound waves) or other signals or energy forms from the stimulator to a garment or other type of electrode or transducer, instead of or in addition to the delivery of electrical stimulation, to improve circulation, enhance healing and/or prevent injury. For example, alternating sequences of noise and electrical stimulation can be delivered to a target body portion of a human or animal subject. In an example embodiment, the stimulator 30 delivers treatment regimen options to the garment electrode 10, including:

a 30-minute cycle with 15 minutes of stimulation at 100 Hz followed by 15 minutes at 10 Hz; and/or

an 8-hour cycle with 10 minutes of stimulation at 80 Hz, followed by 10 minutes at 8 Hz, followed by 40 minutes without stimulation, repeated eight times.

The present invention further comprises a treatment method for delivering energy to a body portion of a human or animal subject. In example embodiments, the treatment is carried out during sleep, when most healing naturally occurs. Without being in any way limited or bound by theory, it is believed that growth hormone activity in the body is naturally more active during resting periods, and that healing therefore takes place more rapidly during these periods. However, bloodflow to the extremities is believed to slow during these periods to maintain core body temperature during inactivity. Delivery of a combination of compression and stimulation to one or more portions of the body, and in particular the extremities, is believed to increase bloodflow in these body portions to enhance the healing process. In example embodiments, the treatment method of the present invention includes debridement of dead or affected tissue, lessening or removing load on the treated area (for example, by using a cane, crutch, walker or wheelchair to offload an affected leg), wound care by cleaning and/or use of antibiotics, and electrical stimulation or otherwise applying energy to enhance circulation in a treated area and/or surrounding tissue under compression, and more preferably under gradient compression. In example forms, the stimulation is applied in cycles, for example by providing stimulation for about twenty minutes in each hour.

While the invention has been described with reference to preferred and example embodiments, it will be understood by those skilled in the art that a number of modifications, additions and deletions are within the scope of the invention, as defined by the following claims.

What is claimed is:

1. A therapeutic garment comprising:
at least one compressive portion; and
at least one electrically conductive portion.

2. The therapeutic garment of claim 1, wherein the at least one compressive portion provides gradient compression when applied to a treated area.

3. The therapeutic garment of claim 1, wherein the at least one compressive portion is integral with the at least one electrically conductive portion.

4. The therapeutic garment of claim 1, comprising a stocking.

5. The therapeutic garment of claim 1, comprising a glove.

6. The therapeutic garment of claim 1, comprising a sleeve.

7. The therapeutic garment of claim 1, at least partially formed of a knit comprising conductive silver nylon fibers.

8. The therapeutic garment of claim 1, comprising a first electrically conductive portion, a second electrically conductive portion, and a non-conductive portion between the first and second electrically conductive portions.

9. A therapeutic garment, at least a portion of which comprises an elastic knit of electrically-conductive fibers, the elastic knit being configured to apply gradient compression when applied to a treated area.

10. The therapeutic garment of claim 9, comprising a first electrically conductive portion, a second electrically conductive portion, and a non-conductive portion between the first and second electrically conductive portions.

11. The therapeutic garment of claim 9, wherein the electrically-conductive fibers comprise silver.

12. The therapeutic garment of claim 9, comprising a stocking.

13. The therapeutic garment of claim 9, comprising a glove.

14. The therapeutic garment of claim 9, comprising a sleeve.

15. A therapeutic garment comprising:
a first conductive portion comprising an electrically-conductive elastic knit material;
a second conductive portion comprising an electrically-conductive elastic knit material; and
a non-conductive portion between the first and second conductive portions;
wherein the elastic knit of the first conductive portion is configured to apply a greater degree of compression to a treated area than the elastic knit of the second conductive portion.

16. The therapeutic garment of claim 15, wherein the electrically-conductive elastic knit material of the first and second conductive portions comprises silver.

17. The therapeutic garment of claim 15, comprising a stocking, wherein the first conductive portion is proximal a toe portion of the stocking and wherein the second conductive portion is proximal a calf or knee portion of the stocking.

18. The therapeutic garment of claim 15, comprising a glove, wherein the first conductive portion is proximal a finger portion of the glove and wherein the second conductive portion is proximal a wrist or forearm portion of the glove.

19. The therapeutic garment of claim 15, comprising a sleeve, wherein the first conductive portion is proximal a first open end of said sleeve and wherein the second conductive portion is proximal a second open end of the sleeve, and wherein the first open end of said sleeve is smaller than the second open end of said sleeve.

20. A method of improving circulation in a human or animal subject, said method comprising:
wearing a compressive garment on a body portion of the subject; and
applying electrical stimulation to at least a portion of the compressive garment.

21. The method of claim 20, wherein the step of wearing a compressive garment on a body portion comprises application of gradient compression to the body portion.

22. The method of claim 20, wherein the electrical stimulation is delivered in a twin-peak monophasic waveform.
